(19) United States
(12) Patent Application Publication
(10) Pub. No.: US 2003/0028976 A1
(43) Pub. Date: Feb. 13, 2003

(54) Compositions for dyeing keratinic fibres, containing paraphenylenediamine derivatives with an azetidinyl group

(76) Inventors: Eric Terranova, Bois Colombes (FR); Aziz Fadli, Chelles (FR)

Correspondence Address: Finnegan Henderson Farabow, Garrett & Dunner, 1300 I Street NW, Washington, DC, 20005, US

(21) Appl. No.: 09/890,856

(22) PCT Filed: Dec. 7, 2000
(86) PCT No.: PCT/FR00/03433

(30) Foreign Application Priority Data
Dec. 8, 1999 (FR) 99/15570

(51) Int. Cl. A61K 7/13

(52) U.S. Cl. 8/405; 8/406

(57) ABSTRACT

The invention relates to novel compositions for the oxidation dyeing of keratin fibers, comprising at least one para-phenylenediamine derivative containing an azetidinyl group as oxidation base, to the dyeing process and to the dyeing kit using this composition, and also to novel para-phenylenediamines containing an azetidinyl group.

The invention relates to novel compositions for the oxidation dyeing of keratin fibres, comprising at least one para-phenylenediamine derivative containing an azetidinyl group as oxidation base, to the dyeing process and to the dyeing kit using this composition, and also to novel para-phenylenediamines containing an azetidinyl group.

It is known practice to dye keratin fibres, and in particular human hair, with dye compositions containing oxidation dye precursors, in particular ortho- or para-phenylenediamines, ortho- or para-aminophenols and heterocyclic compounds such as diaminopyrazole derivatives which are generally referred to as oxidation bases. Oxidation dye precursors, or oxidation bases, are colourless or weakly coloured compounds which, when combined with oxidizing products, can give rise to coloured compounds and dyes by a process of oxidative condensation.

It is also known that the shades obtained with these oxidation bases can be varied by combining them with couplers or colour modifiers, the latter being chosen in particular from aromatic meta-diamines, meta-aminophenols, meta-diphenols and certain heterocyclic compounds.

The variety of molecules used as regards the oxidation bases and the couplers allows a wide range of colours to be obtained.

The so-called “permanent” coloration obtained by means of these oxidation dyes must moreover satisfy a certain number of requirements. Thus it must be able, without toxicological drawbacks, to give shades of the desired intensity and it must be able to withstand external agents (light, bad weather, washing, permanent-waving, perspiration, rubbing).

The dyes must also be able to cover white hair and, lastly, they must be as unselective as possible, i.e. they must give the smallest possible colour differences along the length of the same keratin fibre, which may in fact be differently sensitized (i.e. damaged) between its end and its root.

It has already been proposed, in particular in patent applications WO 98/01106 and EP-A-0 943 614, and also in the Utility Models DE 299 01 593 and DE 299 02 262, to use certain para-phenylenediamine derivatives containing an azetidinyl group as oxidation bases, for the oxidation dyeing of keratin fibres. However, these compounds are not always satisfactory, in particular as regards the intensity of the colorations obtained, their selectivity or their ability to withstand the various attacking factors to which keratin fibres, on which these colorations are carried out, may be subjected.

The Applicant has now discovered, entirely surprisingly and unexpectedly, that certain para-phenylenediamine derivatives containing an azetidinyl group of formulae (I) and/or (II) defined below are not only suitable for use as oxidation bases for the oxidation dyeing of keratin fibres, but also give particularly intense and unselective colorations. Furthermore, they make it possible to obtain dye compositions which give colorations which show good ability to withstand the various attacking factors to which the hair may be subjected.

These discoveries form the basis of the present invention.

A first subject of the invention is thus a composition for the oxidation dyeing of keratin fibres, and in particular of human keratin fibres such as the hair, characterized in that it comprises, in a medium which is suitable for dyeing, at least one oxidation base chosen from para-phenylenediamine derivatives containing an azetidinyl group, of formulae (I) and (II) below, and the addition salts thereof with an acid:

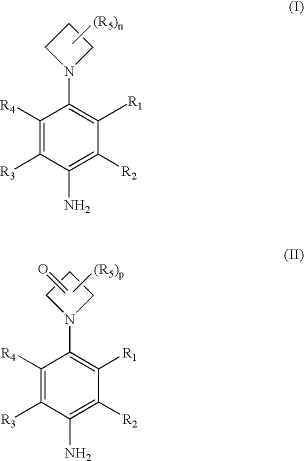

in which:

R₁, R₂, R₃, R₄ and R₅, which may be identical or different, represent a hydrogen atom; a halogen atom; a hydroxyl radical; a C₁-C₆ alkyl radical; a C₂-C₆ alkenyl radical; a C₂-C₆ alkynyl radical; a C₁-6 alkoxy radical; a carbamyl radical; an O—CO—NH₂ radical; an N-(C₁-C₆)alkylcarbamyl radical; an N,N-di(C₁-C₆)-alkylcarbamyl radical; an amino radical; a (C₁-C₆)alkylamino radical; a di(C₁-C₆)alkylamino radical; a (C₁-C₆)alkylcarbonyl radical; a carboxyl radical; a (C₁-C₆) alkylcarboxyl radical; a (C₁-C₆) alkylcarbonyloxy radical; a C₁-C₆ trifluoroalkyl radical; a cyano radical; a (C₁-C₆)alkylthio radical; a formyl radical; a radical CH═NHR₆; or a 5- or 6-membered heterocycle containing from 1 to 3 heteroatoms chosen from oxygen, nitrogen and sulphur;

R₆ represents a C₁-C₆ alkyl radical; an aromatic ring such as, for example, a phenyl ring, or a 5- or 6-membered heteroaromatic ring containing from 1 to 3 heteroatoms chosen from oxygen, nitrogen and sulphur atoms;

n is an integer between 1 and 4 inclusive; preferably between 1 and 3,

p is an integer equal to 1 or 2; it being understood that:

in formula (I), when n=1 and when R₅ represents a hydrogen atom and when one of the radicals R₁ to R4 represents a substituted or unsubstituted amino radical, then at least one of the other radicals R₁ to R₄ is other than a hydrogen atom;

in formula (I), when n=1, and when R₅ represents a hydrogen atom, and when R₂ and R₃ simultaneously represent a hydrogen atom and when one of the radicals R₁ or R₄ also represents a hydrogen atom, a halogen atom, a C₁-C₆ alkyl radical, a C₁-C₆ hydroxyalkyl radical or a (C₁-₆)alkoxy(C₁-C₆)alkyl radical, then the other radical R₁ or R₄ cannot represent a substituted or unsubstituted 5-membered heterocycle.

As mentioned above, the colorations obtained with the oxidation dye composition in accordance with the invention are intense and unselective, and also have excellent properties of withstanding the action of various external agents (light, bad weather, washing, permanent waving, perspiration and rubbing). The oxidation dye compositions in accordance with the invention furthermore make it possible to achieve shades in a very broad range of colours.

In formulae (I) and (II) above, the halogen atoms are chosen from bromine, chlorine, iodine and fluorine, and the expression “C₁-C₆ alkyl” means a linear or branched alkyl chain containing from 1 to 6 carbon atoms which may be substituted with one or more hydroxyl, amino, acylamino, carbamate or ureido radicals, or optionally with a saturated or unsaturated 5- or 6-membered heterocycle. The hydroxyl and/or amino radicals contained in the said alkyl chain may themselves be unsubstituted or substituted with one or more C₁-C₆ alkyl radicals.

Among the para-phenylenediamine derivatives containing an azetidinyl group, of formulae (I) and (II) above, which may be used as oxidation bases in the dye compositions in accordance with the invention, mention may be made in particular of:

4-azetidin-1-ylphenylamine;

1-(4-aminophenyl)azetidine-2-carboxylic acid;

4-azetidin-1-yl-3-methylphenylamine;

1-(4-aminophenyl)azetidine-2-carboxamide;

1-(4-amino-2-methylphenyl)azetidine-2-carboxylic acid;

4-azetidin-1-yl-2-methylphenylamine;

1-(4-amino-3-methylphenyl)azetidine-2-carboxylic acid;

2-(2-amino-5-azetidin-1-ylphenyl)ethanol;

1-[4-amino-3-(2-hydroxyethyl)phenyl]azetidine-2-carboxylic acid;

2-(5-amino-2-azetidin-1-ylphenyl)ethanol;

1-[4-amino-2-(2-hydroxyethyl)phenyl]azetidine-2-carboxylic acid;

1-(5-amino-2-azetidin-1-ylphenyl)ethane-1,2-diol;

1-[4-amino-2-(1,2-dihydroxyethyl)phenyl]azetidine-2-carboxylic acid;

1-(2-amino-5-azetidin-1-ylphenyl)ethane-1,2-diol;

1-[4-amino-3-(1,2-dihydroxyethyl)phenyl]azetidine-2-carboxylic acid;

4-azetidin-1-yl-3-dimethylaminomethylphenylamine;

1-(4-amino-2-dimethylaminomethylphenyl)azetidine-2-carboxylic acid;

4-[3-(2-methoxyethoxy)azetidin-1-yl]phenylamine;

4-[2-(2-methoxyethoxy)azetidin-1-yl]-3-methyl-phenylamine;

4-[3-(2-methoxyethoxy)azetidin-1-yl]-2-methyl-phenylamine;

4-azetidin-1-yl-3-fluorophenylamine;

4-[3-(2-methoxyethoxy)azetidin-1-yl]-3-fluoro-phenylamine;

1-(aminophenyl)azetidine-4-oxo-2-carboxylic acid;

1-(4-aminophenyl)azetidin-3-ol;

1-(4-aminophenyl)-3-methylazetidin-3-ol;

[1-(4-aminophenyl)azetidin-2-yl]methanol;

[1-(4-aminophenyl)-4-hydroxymethylazetidin-2-yl]-methanol and the addition salts thereof with an acid.

Among these para-phenylenediamine derivatives containing an azetidinyl group, of formulae (I) and (II), the ones that are more particularly preferred are:

4-azetidin-1-ylphenylamine;

1-(4-aminophenyl)azetidine-2-carboxylic acid;

1-(4-aminophenyl)azetidine-2-carboxamide;

4-azetidin-1-yl-3-methylphenylamine;

1-(4-amino-2-methylphenyl)azetidine-2-carboxylic acid;

4-azetidin-1-yl-3-dimethylaminomethylphenylamine;

2-(5-amino-2-azetidin-1-ylphenyl)ethanol;

1-[4-amino-2-(2-hydroxyethyl)phenyl]azetidine-2-carboxylic acid;

1-(5-amino-2-azetidin-1-ylphenyl)ethane-1,2-diol;

1-[4-amino-2-(1,2-dihydroxyethyl)phenyl]azetidine-2-carboxylic acid;

1-(2-amino-5-azetidin-1-ylphenyl)ethane-1,2-diol;

1-[4-amino-3-(1,2-dihydroxyethyl)phenyl]azetidine-2-carboxylic acid;

1-(4-aminophenyl)azetidin-3-ol;

1-(4-aminophenyl)-3-methylazetidin-3-ol

[1-(4-aminophenyl)azetidin-2-yl]methanol

[1-(4-aminophenyl)-4-hydroxymethylazetidin-2-yl]methanol; and the addition salts thereof with-an acid.

The para-phenylenediamine derivative(s) containing an azetidinyl group, of formulae (I) and/or (II) in accordance with the invention and/or the addition salt(s) thereof with an acid preferably represent from 0.0005% to 12% by weight approximately relative to the total weight of the dye composition, and even more preferably from 0.005% to 6% by weight approximately relative to this weight.

The medium which is suitable for the dyeing (or the support) generally consists of water or of a mixture of water and at least one organic solvent in order to dissolve the compounds which would not be sufficiently soluble in water. By way of organic solvent, mention may be made, for example, of C₁-C₄ lower alkanols such as ethanol and isopropanol; glycerol; glycols and glycol ethers such as 2-butoxyethanol, propylene glycol, propylene glycol monomethyl ether, diethylene glycol monoethyl ether and monomethyl ether, and aromatic alcohols such as benzyl alcohol or phenoxyethanol, similar products and mixtures thereof.

The solvents can be present in proportions preferably of between 1% and 40% by weight approximately relative to the total weight of the dye composition, and even more preferably between 5% and 30% by weight approximately.

The pH of the dye composition in accordance with the invention is generally between 3 and 12 approximately and preferably between 5 and 11 approximately. It may be adjusted to the desired value with the aid of acidifying or basifying agents commonly used in the dyeing of keratin fibres or else with the aid of conventional buffer systems.

Among the acidifying agents, mention may be made, for example, of inorganic or organic acids such as hydrochloric acid, orthophosphoric acid, sulphuric acid, carboxylic acids such as acetic acid, tartaric acid, citric acid or lactic acid, and sulphonic acids.

Among the basifying agents, mention may be made, for example, of aqueous ammonia, alkaline carbonates, alkanolamines such as mono-, di- and triethanolamines and derivatives thereof, sodium hydroxide, potassium hydroxide and the compounds of formula (III) below:

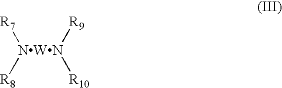

in which W is a propylene residue optionally substituted with a hydroxyl group or a C₁-C₄ alkyl radical; R₇, R₈, R₉ and R₁₀, which may be identical or different, represent a hydrogen atom or a C₁-C₄ alkyl or C₁-C₄ hydroxyalkyl radical.

According to one preferred embodiment, the oxidation dye composition in accordance with the invention also contains one or more couplers so as to modify the shades obtained or to enrich them with glints by using the compounds of formulae (I) and/or (II).

The couplers which may be used in the oxidation dye compositions in accordance with the invention may be chosen from the couplers used conventionally in oxidation dyeing, and among which mention may be made in particular of meta-phenylenediamines, meta-aminophenols, meta-diphenols and heterocyclic couplers.

These couplers are chosen more particularly from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-methyl-3-amino-6-methylphenol, 3,5-diamino-2,6-dimethoxypyridine, 6-hydroxybenzomorpholine, 1,2′-hydroxyethylamino-3′-methylenedioxybenzene and 1-methyl-2,6-bis(2′-hydroxyethylamino)benzene, and the addition salts thereof with an acid.

When they are present, the coupler(s) preferably represent(s) from 0.0001% to 10% by weight approximately relative to the total weight of the dye composition and even more preferably from 0.005% to 5% by weight approximately relative to this weight.

The dye composition in accordance with the invention may also contain, in addition to the compounds of formulae (I) and/or (II) as defined above and the couplers defined above, at least one additional oxidation base, which may be chosen from the oxidation bases conventionally used in oxidation dyeing and among which mention may be made in particular of para-phenylenediamines other than the compounds of formulae (I) and (II) in accordance with the invention, bis(phenyl)alkylenediamines, para-aminophenols, ortho-aminophenols and heterocyclic bases, and the addition salts thereof with an acid.

Among the para-phenylenediamines, mention may be made more particularly, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamlne, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine and N-(β-methoxyethyl)-para-phenylenediamine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the addition salts thereof with an acid are most particularly preferred.

Among the bis(phenyl)alkylenediamines, mention may be made more particularly, by way of example, of N,N′-bis(β-hydroxyethyl)-N,N′-bis(4′-aminophenyl)-1,3-diaminopropanol, N,N′-bis(β-hydroxyethyl)-N,N′-bis(4-aminophenyl)ethylenediamine, N,N′-bis(4-aminophenyl)tetramethylenediamine, N,N′-bis(β-hydroxyethyl)-N,N′-bis(4-aminophenyl)-tetramethylenediamine, N,N′-bis(4-methylaminophenyl)-tetramethylenediamine, N,N′-bis(ethyl)-N,N′-bis(4′-amino-3′-methylphenyl)ethylenediamine and 1,8-bis(2,5-diaminophenoxy)-3,5-dioxaoctane, and the addition salts thereof with an acid.

Among the para-aminophenols, mention may be made more particularly, by way of example, of paraaminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols, mention may be made more particularly, by way of example, of 2-aminophenol, 2-amino-5-methylphenol, 2-amino-6-methylphenol and 5-acetamido-2-aminophenol, and the addition salts thereof with an acid.

Among the heterocyclic bases, mention may be made more particularly, by way of example, of pyridine derivatives, pyrimidine derivatives and pyrazole derivatives.

Among the pyridine derivatives, mention may be made more particularly of the compounds described, for example, in patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives, mention may be made more particularly of the compounds described, for example, in patents DE 2 359 399; JP 88-169 571; JP 05 163 124; EP 0 770 375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048, and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]-pyrimidin-3-yl)(2-hydroxyethyl)amino]ethanol, 5,6-dimethylpyrazolo [1,5-apyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, the addition salts thereof with an acid, and the tautomeric forms thereof, when a tautomeric equilibrium exists.

Among the pyrazole derivatives, mention may be made more particularly of the compounds described in patents DE 3 843 892, DE 4 133 957 and patent applications WO 94/08969, WO 94/08970, FR-A-2,733,749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

When they are used, the additional oxidation base(s) preferably represent(s) from 0.0005% to 12% by weight approximately relative to the total weight of the dye composition, and even more preferably from 0.005% to 6% by weight approximately relative to this weight.

In general, the addition salts with an acid which may be used in the context of the dye compositions of the invention (compounds of formulae (I) and (II), couplers and additional oxidation bases) are chosen in particular from the hydrochlorides, hydrobromides, sulphates, citrates, succinates, tartrates, lactates, phosphates and acetates.

The dye composition in accordance with the invention may also contain one or more direct dyes which may be chosen in particular from dyes of the nitrobenzene series.

The dye composition in accordance with the invention can also contain various adjuvants used conventionally in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, antioxidants, penetration agents, sequestering agents, fragrances, buffers, dispersing agents, conditioners, such as, for example, volatile or non-volatile silicones, which are modified or unmodified, film-forming agents, ceramides, preserving agents and opacifiers.

Needless to say, a person skilled in the art will take care to select this or these optional complementary compound(s) such that the advantageous properties intrinsically associated with the oxidation dye composition in accordance with the invention are not, or not substantially, adversely affected by the addition or additions envisaged.

The dye composition in accordance with the invention can be in various forms, such as in the form of liquids, creams or gels or any other form which is suitable for dyeing keratin fibres, and in particular human hair.

A subject of the invention is also a process for dyeing keratin fibres, and in particular human keratin fibres such as the hair, using the dye composition as defined above.

According to this process, at least one dye composition as defined above is applied to the fibres, the colour being developed at acidic, neutral or alkaline pH with the aid of an oxidizing agent which is added to the dye composition just at the time of use, or which is present in an oxidizing composition that is applied simultaneously or sequentially.

According to one preferred embodiment of the dyeing process of the invention, the dye composition described above is preferably mixed, at the time of use, with an oxidizing composition containing, in a medium which is suitable for dyeing, at least one oxidizing agent present in an amount which is sufficient to develop a coloration. The mixture obtained is then applied to the keratin fibres and is left to stand on them for about 3 to 50 minutes, preferably about 5 to 30 minutes, after which the fibres are rinsed, washed with shampoo, rinsed again and dried.

The oxidizing agent present may be chosen from the oxidizing agents conventionally used for the oxidation dyeing of keratin fibres, and among which mention may be made of hydrogen peroxide, urea peroxide, alkali metal bromates, persalts such as perborates and persulphates, and enzymes, among which mention may be made of peroxidases, 2-electron oxidoreductases such as uricases, and 4-electron oxygenases such as laccases. Hydrogen peroxide is particularly preferred.

The pH of the oxidizing composition containing the oxidizing agent as defined above is such that, after mixing it with the dye composition, the pH of the resulting composition applied to the keratin fibres preferably ranges between 3 and 12 approximately, and even more preferably between 5 and 11. It is adjusted to the desired value by means of acidifying or basifying agents usually used for the dyeing of keratin fibres and as defined above.

The oxidizing composition as defined above can also contain various adjuvants conventionally used in compositions for dyeing the hair and as defined above.

The composition which is finally applied to the keratin fibres can be in various forms, such as in the form of liquids, creams or gels or any other form which is suitable for dyeing keratin fibres, and in particular human hair.

Another subject of the invention is a multi-compartment device or dyeing “kit” or any other multi-compartment packaging system, a first compartment of which contains the dye composition as defined above and a second compartment of which contains the oxidizing composition as defined above. These devices may be equipped with a means for applying the desired mixture to the hair, such as the devices described in patent FR-2 586 913 in the name of the Applicant.

Certain para-phenylenediamine derivatives containing an azetidinyl group, of formulae (I) and (II), used as oxidation bases in the context of the present invention are novel and, in this respect, constitute another subject of the invention.

These novel para-phenylenediamine derivatives containing an azetidinyl group, and also the addition salts thereof with an acid, correspond to the formulae (I′) and (II′) below:

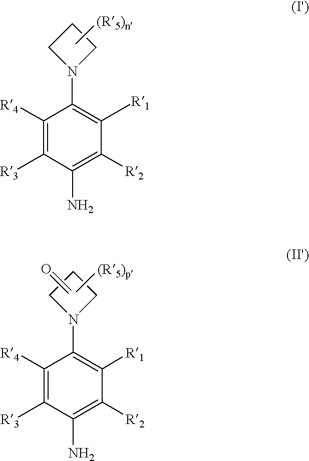

in which:

R′₁, R′₂, R′₃, R′₄ and R′₅, which may be identical or different, represent a hydrogen atom; a halogen atom; a hydroxyl radical; a C₁-C₆ alkyl radical; a C₂-C₆ alkenyl radical; a C₂-C₆ alkynyl radical; a C₁-C₆ alkoxy radical; a carbamyl radical; a carboxamide radical; an N-(C₁-6)alkylcarbamyl radical; an N,N-di(C₁-C₆)-alkylcarbamyl radical; an amino radical; a (C₁-C₆)alkylamino radical; a di(C₁-C₆)alkylamino radical; a (C₁-C₆)alkylcarbonyl radical; a carboxyl radical; a (C₁-₆)alkylcarboxyl radical; a (C₁-6)alkylcarbonyloxy radical; a C₁-C₆ trifluoroalkyl radical; a cyano radical; a (C₁-C₆)alkylthio radical; a formyl radical; a radical CH═NHR′_{6;} or a 5- or 6-membered heterocycle containing from 1 to 3 heteroatoms chosen from oxygen, nitrogen and sulphur;

R′₆ represents a C₁-C₆ alkyl radical; an aromatic ring such as, for example, a phenyl ring, or a 5- or 6-membered heteroaromatic ring containing from 1 to 3 heteroatoms chosen from oxygen, nitrogen and sulphur atoms;

n′ is an integer between 1 and 4 inclusive; preferably between 1 and 3,

p′ is an integer equal to 1 or 2; it being understood that:

in formula (I), when n′=1 and when R′₅ represents a hydrogen atom and when one of the radicals R′₁ to R′₄ represents a substituted or unsubstituted amino radical, then at least one of the other radicals R′₁ to R′₄ is other than a hydrogen atom;

in formula (I), when n′=1, and when R′₅ represents a hydrogen atom, and when R′₂ and R′₃ simultaneously represent a hydrogen atom and when one of the radicals R′₁, or R′₄ also represents a hydrogen atom, a halogen atom, a C₁-C₆ alkyl radical, a C₁-C₆ hydroxyalkyl radical or a (C₁-C₆)alkoxy(C₁-C₆)alkyl radical, then the other radical R′₁ or R′₄ cannot represent a substituted or unsubstituted 5-membered heterocycle;

with the exclusion of:

4-azetidin-1-yl-3-fluorophenylamine;

3-fluoro-4-[3-(2-methoxyethoxy)azetidin-1-yl]-phenylamine;

diethyl 1-(4-aminophenyl)-2-oxoazetidine-3,3-dicarboxylate;

diethyl 1-(4-aminophenyl)-2-[1,3]dioxolan-2-yl-4-oxoazetidine-3,3-dicarboxylate;

1-(4-aminophenyl)-4-oxoazetidine-2-carboxylic acid;

methyl 1-(4-aminophenyl)-4-oxoazetidin-2-yl-methanesulphonate;

methyl 1-(4-aminophenyl)-4-oxoazetidin-2-yltoluene-4-sulphonate.

The compounds specifically excluded from the subject of formulae (I′) and (II′) above are known in the pharmaceutical field, in particular as antimicrobial agents (see in particular patent application WO 99/12914 and Nicolaus et al., Helvetica Chim. Acta. Vol 48, Issue No. 8, (1965), No. 200-201, pages 1867-1885).

Among the compounds of formulae (I′) and (II′) above, mention may be made in particular of:

4-azetidin-1-ylphenylamine;

1-(4-aminophenyl)azetidine-2-carboxylic acid;

1-(4-aminophenyl)azetidine-2-carboxamide;

4-azetidin-1-yl-3-methylphenylamine;

1-(4-amino-2-methylphenyl)azetidine-2-carboxylic acid;

4-azetidin-1-yl-2-methylphenylamine;

1-(4-amino-3-methylphenyl)azetidine-2-carboxylic acid;

2-(2-amino-5-azetidin-1-ylphenyl)ethanol;

1-[4-amino-3-(2-hydroxyethyl)phenyl]azetidine-2-carboxylic acid;

2-(5-amino-2-azetidin-1-ylphenyl)ethanol;

1-[4-amino-2-(2-hydroxyethyl)phenyl]azetidine-2-carboxylic acid;

1-(5-amino-2-azetidin-1-ylphenyl)ethane-1,2-diol;

1-[4-amino-2-(1,2-dihydroxyethyl)phenyl]azetidine-2-carboxylic acid;

1-(2-amino-5-azetidin-1-ylphenyl)ethane-1,2-diol;

1-[4-amino-3-(1,2-dihydroxyethyl)phenyl]azetidine-2-carboxylic acid;

4-azetidin-1-yl-3-dimethylaminomethylphenylamine;

1-(4-amino-2-dimethylaminomethylphenyl)azetidine-2-carboxylic acid;

4-[3-(2-methoxyethoxy)azetidin-1-yl]phenylamine;

4-[2-(2-methoxyethoxy)azetidin-1-yl]-3-methyl-phenylamine;

4-[3-(2-methoxyethoxy)azetidin-1-yl]-2-methyl-phenylamine;

1-(4-aminophenyl)azetidin-3-ol

1-(4-aminophenyl)-3-methylazetidin-3-ol

1-(4-aminophenol)azetidin-2-yl]methanol

[1-(4-aminophenyl)-4-hydroxymethylazetidin-2-yl]-methanol

and the addition salts thereof with an acid.

The addition salts with an acid of the compounds of formulae (I′) and (II′) may be chosen from the hydrochlorides, hydrobromides, sulphates, citrates, succinates, tartrates, lactates, phosphates and acetates.

The compounds of formulae (I′) and (II′) in accordance with the invention may be prepared according to the following synthetic scheme:

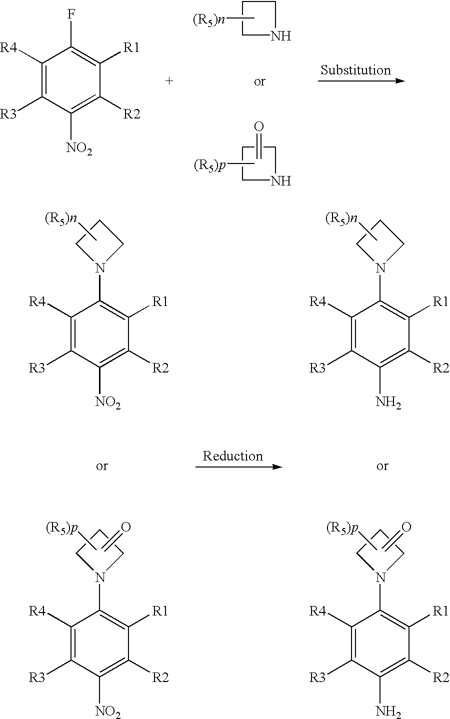

For the substitution step, methods that are well known in the literature which consist, for example, in carrying out a substitution reaction of an amine of azetidinyl type on a benzene derivative of p-halonitrobenzene type such as, for example, a p-fluoro-nitrobenzene, may be used. Conventional substitution methods as described in the literature are used. The syntheses may be inspired, for example, from the methods described in the following references:

Tetrahedron, 51(22), 6167, 1995

Synthesis, 12, 1147, 1990

J. Med. Chem., 33(7), 2045, 1990

J. Chem. Soc. Perkin Trans. 1, 6, 1331, 1988

J. Chem. Soc. Perkin Trans. 1, 3, 549, 1988

Liebigs Ann. Chem., 4, 343, 1988

Chem. Pharm. Bull., 33(5), 1826, 1985

The nitro derivatives thus obtained may be reduced according to known methods: see in particular R. Hemmer and W. Lürken in Houben-Weyl, “Methoden der Organischen Chemie”, Vol. E16d, p. 815ff. It will be preferred to use metals such as palladium (Pd), platinum (Pt) or nickel (Ni) in the presence of a hydrogen donor such as ammonium formate, formic acid or cyclohexene instead of hydrogen (S. Ram. R. E. Ehrenkaufer, Synthesis, 91, 1988). Metals such as zinc (Zn), tin (Sn) or iron (Fe) may also be used, in acidic medium such as aqueous hydrochloric acid or aqueous acetic acid, optionally with addition of an organic solvent such as methanol, ethanol or tetrahydrofuran.

Another subject of the invention is the use of the para-phenylenediamine derivatives containing an azetidinyl group, of formulae (I), (II), (I′) and (II′) above, as oxidation bases for the oxidation dyeing of keratin fibres, and in particular of human keratin fibres such as the hair.

The examples which follow are intended to illustrate the invention without, however, limiting its scope.

### EXAMPLES

### Example 1

### Synthesis of 4-azetidin-1-ylphenylamine

a) First step: preparation of 1-(4-nitrophenyl)-azetidine

20 ml of ethanol and 20 mmol of 1-fluoro-4-nitrobenzene were introduced into a three-necked round-bottomed flask on which was mounted a condenser, an addition funnel and a thermometer, and 22 mmol of azetidine were added over 10 minutes. Stirring was continued for 1 hour.

The crystalline product was filtered off, spin-filtered, washed with alcohol and dried under vacuum.

The expected product was recovered in the form of needles of a bright yellow compound, in a yield of 73%.

b) Second step: reduction of 1-(4-nitrophenyl)azetidine

2.5 g of 1-(4-nitrophenyl)azetidine obtained above in the preceding step were reduced under hydrogen-transfer conditions in 30 ml of ethanol and 30 ml of cyclohexene in the presence of 1 g of palladium-on-charcoal containing 5% water.

The reaction medium was refluxed for one hour, the catalyst was removed by filtration and two equivalents of hydrochloric acid were added at zero degrees. The reduced compound crystallized on dilution with diisopropyl ether. After drying, the expected product was recovered in the form of a white compound, in a yield of 93%.

The ¹H NMR analysis (CD₃OD) δ ppm was as follows: 2.59 (2H, multiplet); 4.43 (4H, triplet); 7.48 (4H, multiplet)

### Example 2

### Synthesis of the tartrate salt of 1-(4-aminophenyl)-3-hydroxy-3-methylazetidine

Step A: preparation of 1-chloro-2,3-epoxy-2-methyl-propane

73.5 ml (0.75 mol) of methallyl chloride are added to 375 ml of water in a 1-liter reactor, followed by addition of 133.5 g (1 eq) of N-bromosuccinimide with vigorous stirring at room temperature.

After leaving overnight, the mixture is cooled to 10° C. and aqueous 50% sodium hydroxide (0.75 mol) is added at a rate such that the temperature is maintained at between 20 and 25° C.

After 2 hours without stirring, the lower organic phase is separated out and dried over sodium sulphate (10 g), and the organic phase is concentrated. 53.3 g (67%) of crude product are obtained.

Separately, the aqueous phase is extracted with dichloromethane and the extracts are dried over sodium sulphate and concentrated. A further 23 g of crude product are obtained. The combined crude product is distilled (60° C./65 mb) to give finally 37.8 g of a colourless liquid, i.e. a yield of 47%.

Step B: preparation of 1-diphenylmethyl-3-hydroxy-3-methylazetidine hydrochloride

28 g (0.263 mol) of 1-chloroepoxy-2-methyl-propane are added to a solution of 48.2 g (1 eq) of diphenylmethylamine dissolved in 120 ml of methanol. The mixture is stirred for 3 days at room temperature and then for 3 days at reflux. The resulting mixture is cooled and the white precipitate obtained is then filtered off. This product is washed with acetone and then dried under vacuum over potassium hydroxide to give 46.2 g (61%) of 1-diphenylmethyl-3-hydroxy-3-methylazetidine hydrochloride.

Elemental analysis (C₁₇H₂₀NOCl; MW=289.804)

| | % C | % H | % N | % O | % Cl |
|---|---|---|---|---|---|
| Theoretical | 70.46 | 6.96 | 4.83 | 5.52 | 12.23 |
| Found | 70.36 | 6.96 | 5.19 | 5.89 | 12.04 |

Step C: preparation of 1-(4-nitrophenyl)-3-hydroxy-3-methylazetidine

36 g (0.124 mol) of 1-diphenylmethyl-3-hydroxy-3-methylazetidine are dissolved in 450 ml of methanol and 15 g of Pd(OH)₂ (20% by weight) are then added. The mixture is placed under a hydrogen pressure of 10 bar at a temperature of 25° C. for 2 hours. The catalyst is then filtered off and the solvent is evaporated until a two-phase oil is obtained. The oil obtained is diluted in 100 ml of N-methylpyrrolidone and 17.5 g (1 eq) of 4-fluoronitrobenzene are added, followed by 42.9 g (2.5 eq) of potassium carbonate. The mixture is heated at 95° C. for 5 hours and is then poured into 1l of water. The yellow precipitate obtained is filtered off and washed with water. The crude product is dried under vacuum over potassium hydroxide and is re-slurried in petroleum ether. This product is filtered off, washed with petroleum ether and dried to give 22 g (85%) of 1-(4-nitrophenyl)-3-hydroxy-3-methylazetidine.

Elemental analysis (C₁₀H₁₂NO₃; MW=208.216)

| | % C | % H | % N | % O |
|---|---|---|---|---|
| Theoretical | 57.69 | 5.81 | 13.45 | 23.05 |
| Found | 57.29 | 5.72 | 13.27 | 23.08 |

Step D: preparation of the tartrate salt of 1-(4-aminophenyl)-3-hydroxy-3-methylazetidine

20.8 g (0.1 mol) of 1-(4-nitrophenyl)-3-hydroxy-3-methylazetidine suspended in 200 ml of ethanol are introduced into a hydrogenator in the presence of 4 g of wet palladium-on-charcoal. The mixture is hydrogenated under a pressure of 6 bar at room temperature for a period of 3 hours.

After filtering off the catalyst under nitrogen, the filtrate is collected in a solution of 96° ethanol containing 15.1 g (1 eq) of L-tartaric acid. The precipitate is filtered off, washed with isopropanol and dried under vacuum to give 25.9 g (79%) of tartrate salt of 1-(4-aminophenyl)-3-hydroxy-3-methylazetidine.

Mass spectrum (TSQ 700; ESI-ID): m/z=179 (MH)+

Elemental analysis (MW=328.319; C₁₄H₂₀N₂O₇)

| | % C | % H | % N | % O |
|---|---|---|---|---|
| Theoretical | 51.22 | 6.14 | 8.53 | 34.11 |
| Found | 50.61 | 6.44 | 8.23 | 34.74 |

### Example 3

### Preparation of the Tartrate Salt of 1-(4-aminophenyl)-3-hydroxyazetidine

### Step 1: Preparation of 1-diphenylmethyl-3-hydroxy-azetidine hydrochloride

This compound is obtained according to the procedure of Example 2, Step B, starting with 88 g (0.480 mol) of diphenylmethylamine and 1 equivalent of epichlorohydrin. 68 g of 1-diphenylmethyl-3-hydroxy-azetidine hydrochloride are obtained, i.e. a yield of 51%.

### Step 2: Preparation of 1-(4-nitrophenyl)-3-hydroxy-azetidine

According to the procedure of Example 2, Step C, starting with 50 g (0.181 mol) of 1-diphenyl-3-hydroxyazetidine hydrochloride, 21.2 g of 1-(4-nitro-phenyl)-3-hydroxyazetidine are obtained, i.e. a yield of 63%.

### Step 3: Preparation of the tartrate salt of 1-(4-aminophenyl)-3-hydroxyazetidine

According to the procedure of Example 2, Step D, starting with 80 g (0.103 mol) of 1-(4-nitrophenyl)-3-hydroxyazetidine, 8.5 g of 1-(4-aminophenyl)-3-hydroxyazetidine are obtained, i.e. a yield of 27%. Mass spectrum (TSQ 700; ESI-ID): m/z=165 (MH)+

### Examples 4 to 17

### Dye Compositions

The dye compositions below in accordance with the invention were prepared (contents in grams):

| EXAMPLES | 4 | 5 | 6 | 7 | 8 | 9 | 10 | 11 | 12 | 13 |
|---|---|---|---|---|---|---|---|---|---|---|
| 4-azetidin-1-ylphenylamine | 6 × 10⁻³ | 6 × 10⁻³ | 6 × 10⁻³ | 6 × 10⁻³ | 6 × 10⁻³ | — | — | — | — | — |
| dihydrochloride | mol | mol | mol | mol | mol | | | | | |
| 1-(4-aminophenyl)-3- | — | — | — | — | — | 6 × 10⁻³ | 6 × 10⁻³ | 6 × 10⁻³ | 6 × 10⁻³ | 6 × 10⁻³ |
| hydroxy-acetidine tartrate | | | | | | mol | mol | mol | mol | mol |
| Resorcinol | — | 0.66 | — | — | — | — | 0.66 | — | — | — |
| m-aminophenol | — | — | 0.65 | — | — | — | — | 0.65 | — | — |
| 1-β-hydroxyethyloxy-2,4- | — | — | — | 1.45 | — | — | — | — | 1.45 | — |
| diamino-benzene | | | | | | | | | | |
| dihydrochloride | | | | | | | | | | |
| 1-methyl-4-aminophenol | — | — | — | — | 0.74 | — | — | — | — | 0.74 |
| Dye support | (*) | (*) | (*) | (*) | (*) | (*) | (*) | (*) | (*) | (*) |
| Demineralized water qs | 100 g | 100 g | 100 g | 100 g | 100 g | 100 g | 100 g | 100 g | 100 g | 100 g |

| EXAMPLES | 14 | 15 | 16 | 17 |
|---|---|---|---|---|
| [1-(4-aminophenyl)-3- | 6 × 10⁻³ | 6 × 10⁻³ | 6 × 10⁻³ | 6 × 10⁻³ |
| hydroxy-3-methylazetidine | mol | mol | mol | mol |
| Resorcinol | — | — | 0.66 | — |
| 1-methyl-4-aminophenol | — | 0.74 | — | — |
| 1-β-hydroxyethyloxy-2,4- | — | — | — | 1.45 |
| diaminobenzene dihydrochloride | | | | |
| Dye support | (*) | (*) | (*) | (*) |
| Demineralized water qs | 100 g | 100 g | 100 g | 100 g |

Dye support used in the above examples.

| | | |
|---|---|---|
| Oleyl alcohol polyglycerolated with 4 mol | 5.7 | g A.M. |
| of glycerol, containing 78% active material | | |
| (A.M.) | | |
| Oleyl alcohol polyglycerolated with 2 mol | 4.0 | g |
| of glycerol | | |
| Oleic acid | 3.0 | g |
| Oleylamine containing 2 mol of ethylene | 7.0 | g |
| oxide, sold under the name Ethomeen 012 | | |
| by the company Akzo | | |
| Diethylaminopropyl laurylaminosuccinamate, | 3.0 | g A.M. |
| sodium salt, containing 55% A.M. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulphite as an aqueous | 0.46 | g A.M. |
| solution containing 35% A.M. | | |
| Ammonium acetate | 0.8 | g |
| Antioxidant, sequestering agent | qs | |
| Fragrance, preserving agent | qs | |
| Aqueous ammonia solution containing | 10.0 | g. |
| 20% NH₃ | | |

At the time of use, each of the compositions is mixed weight-for-weight with 20-volumes aqueous hydrogen peroxide solution (6% by weight), of pH 3. A mixture of pH 9.8 is obtained.

This mixture is applied to natural (BN) or permanent-waved (BP) grey hair containing 90% white hairs, for 30 minutes.

After rinsing, washing with shampoo, rinsing and drying, each lock is evaluated before and after dyeing in the L*a*b* system, using a Minolta CM 2002® spectrophotometer (illuminant D65).

In the L*a*b* system, the three parameters respectively denote the intensity (L*), the shade (a*) and the saturation (b*). According to this system, the higher the value of L, the lighter or less intense the colour. Conversely, the lower the value of L, the darker or more intense the colour. a* and b* indicate two colour axes; a* indicates the green/red colour axis and b* the blue/yellow colour axis.

The results are given in the table below.

| | L* | a* | b* | | L* | a* | b* |
|---|---|---|---|---|---|---|---|
| Ex. 4/BN | 40.15 | 2.6 | 6.48 | Ex. 4/BP | 30.89 | 3.82 | 4.02 |
| Ex. 5/BN | 37.7 | 4.22 | 7.74 | Ex. 5/BP | 30.69 | 4.95 | 7.24 |
| Ex. 6/BN | 34.18 | 2.08 | −0.35 | Ex. 6/BP | 27.35 | 2.34 | −1.13 |
| Ex. 7/BN | 27.39 | −0.49 | −12.39 | Ex. 7/BP | 22.05 | 1.33 | −9.77 |
| Ex. 8/BN | 32.12 | 9.39 | −5.19 | Ex. 8/BP | 24.67 | 8.51 | −6.75 |
| Ex. 9/BN | 42.14 | 3.89 | 6.29 | Ex. 9/BP | 33.60 | 4.57 | 3.56 |
| Ex. 10/BN | 38.39 | 5.28 | 9.84 | Ex. 10/BP | 29.88 | 5.49 | 7.85 |
| Ex. 11/BN | 32.03 | 2.62 | −1.01 | Ex. 11/BP | 25.61 | 2.05 | −0.24 |
| Ex. 12/BN | 24.06 | 1.76 | −12.70 | Ex. 12/BP | 20.25 | 2.41 | −8.18 |
| Ex. 13/BN | 28.34 | 10.65 | −6.20 | Ex. 13/BP | 22.49 | 9.15 | −7.04 |
| Ex. 14/BN | 41.46 | 3.20 | 5.67 | Ex. 14/BP | 42.44 | 3.04 | 4.68 |
| Ex. 15/BN | 39.50 | 4.62 | 8.70 | Ex. 15/BP | 30.08 | 5.35 | 7.67 |
| Ex. 16/BN | 32.06 | 9.92 | −5.58 | Ex. 16/BP | 33.79 | 10.54 | −5.50 |
| Ex. 17/BN | 23.19 | 1.63 | −13.18 | Ex. 17/BP | 24.93 | 1.09 | −12.41 |

1. Composition for the oxidation dyeing of keratin fibres, and in particular of human keratin fibres such as the hair, characterized in that it comprises, in a medium which is suitable for dyeing, at least one oxidation base chosen from para-phenylenediamine derivatives containing an azetidinyl group, of formulae (I) and (II) below, and the addition salts thereof with an acid: 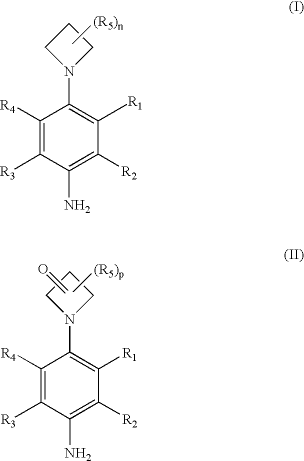
in which:
R₁, R₂, R₃, R₄ and R₅, which may be identical or different, represent a hydrogen atom; a halogen atom; a hydroxyl radical; a C₁-C₆ alkyl radical; a C₂-C₆ alkenyl radical; a C₂-C₆ alkynyl radical; a C₁-C₆ alkoxy radical; a carbamyl radical; a carboxamide radical; an N-(C₁-C₆)alkylcarbamyl radical; an N,N-di(C₁-C₆)-alkylcarbamyl radical; an amino radical; a (C₁-C₆)alkylamino radical; a di(C₁-C₆)alkylamino radical; a (C₁-C₆)alkylcarbonyl radical; a carboxyl radical; a (C₁-C₆) alkylcarboxyl radical; a (C₁-C₆) alkylcarbonyloxy radical; a C₁-C₆ trifluoroalkyl radical; a cyano radical; a (C₁-C₆)alkylthio radical; a formyl radical; a radical CH═NHR₆; or a 5- or 6-membered heterocycle containing from 1 to 3 heteroatoms chosen from oxygen, nitrogen and sulphur;
R₆ represents a C₁-₆ alkyl radical; an aromatic ring such as, for example, a phenyl ring, or a 5- or 6-membered heteroaromatic ring containing from 1 to 3 heteroatoms chosen from oxygen, nitrogen and sulphur atoms;
n is an integer between 1 and 4 inclusive;
p is an integer equal to 1 or 2; it being understood that:
in formula (I), when n=1 and when R₅ represents a hydrogen atom and when one of the radicals R₁ to R₄ represents a substituted or unsubstituted amino radical, then at least one of the other radicals R₁ to R₄ is other than a hydrogen atom;
in formula (I), when n=1, and when R₅ represents a hydrogen atom, and when R₂ and R₃ simultaneously represent a hydrogen atom and when one of the radicals R₁ or R₄ also represents a hydrogen atom, a halogen atom, a C₁-C₆ alkyl radical, a C₁-C₆ hydroxyalkyl radical or a (C₁-C₆)alkoxy(C₁-C₆)alkyl radical, then the other radical R₁ or R₄ cannot represent a substituted or unsubstituted 5-membered heterocycle.

2. Composition according to claim 1, in which n is between 1 and 3.

3. Composition according to claim 1 or 2, characterized in that, in formulae (I) and (II), the halogen atoms are chosen from bromine, chlorine, iodine and fluorine.

4. Compositions according to claim 1, 2 or 3, characterized in that the para-phenylenediamine derivative(s) containing an azetidinyl group, of formula (I) or (II), are chosen from:
4-azetidin-1-ylphenylamine;
1-(4-aminophenyl)azetidine-2-carboxylic acid;
4-azetidin-1-yl-3-methylphenylamine;
1-(4-aminophenyl)azetidine-2-carboxamide;
1-(4-amino-2-methylphenyl)azetidine-2-carboxylic acid;
4-azetidin-1-yl-2-methylphenylamine;
1-(4-amino-3-methylphenyl)azetidine-2-carboxylic acid;
2-(2-amino-5-azetidin-1-ylphenyl)ethanol;
1-[4-amino-3-(2-hydroxyethyl)phenyl]azetidine-2-carboxylic acid;
2-(5-amino-2-azetidin-1-ylphenyl)ethanol;
1-[4-amino-2-(2-hydroxyethyl)phenyl]azetidine-2-carboxylic acid;
1-(5-amino-2-azetidin-1-ylphenyl)ethane-1,2-diol;
1-[4-amino-2-(1,2-dihydroxyethyl)phenyl]azetidine-2-carboxylic acid;
1-(2-amino-5-azetidin-1-ylphenyl)ethane-1,2-diol;
1-[4-amino-3-(1,2-dihydroxyethyl)phenyl]azetidine-2-carboxylic acid;
4-azetidin-1-yl-3-dimethylaminomethylphenylamine;
1-(4-amino-2-dimethylaminomethylphenyl)azetidine-2-carboxylic acid;
4-[3-(2-methoxyethoxy)azetidin-1-yl]phenylamine;
4-[2-(2-methoxyethoxy)azetidin-1-yl]-3-methyl-phenylamine;
4-[3-(2-methoxyethoxy)azetidin-1-yl]-2-methyl-phenylamine;
4-azetidin-1-yl-3-fluorophenylamine;
4-[3-(2-methoxyethoxy)azetidin-1-yl]-3-fluoro-phenylamine;
1-(aminophenyl)azetidine-4-oxo-2-carboxylic acid;
1-(4-aminophenyl)azetidin-3-ol
1-(4-aminophenyl)-3-methylazetidin-3-ol
[1-(4-aminophenyl)azetidin-2-yl]methanol
[1-(4-aminophenyl)-4-hydroxymethylazetidin-2-yl]-methanol
and the addition salts thereof with an acid.

5. Composition according to claim 4, characterized in that the para-phenylenediamine derivative(s) containing an azetidinyl group, of formula (I) or (II), are chosen from:
4-azetidin-1-ylphenylamine;
1-(4-aminophenyl)azetidine-2-carboxylic acid;
1-(4-aminophenyl)azetidine-2-carboxamide;
4-azetidin-1-yl-3-methylphenylamine;
1-(4-amino-2-methylphenyl)azetidine-2-carboxylic acid;
4-azetidin-1-yl-3-dimethylaminomethylphenylamine;
2-(5-amino-2-azetidin-1-ylphenyl)ethanol;
1-[4-amino-2-(2-hydroxyethyl)phenyl]azetidine-2-carboxylic acid;
1-(5-amino-2-azetidin-1-ylphenyl)ethane-1,2-diol;
1-[4-amino-2-(1,2-dihydroxyethyl)phenyl]azetidine-2-carboxylic acid;
1-(2-amino-5-azetidin-1-ylphenyl)ethane-1,2-diol;
1-[4-amino-3-(1,2-dihydroxyethyl)phenyl]azetidine-2-carboxylic acid;
1-(4-aminophenyl)azetidin-3-ol
1-(4-aminophenyl)-3-methylazetidin-3-ol
[1-(4-aminophenyl)azetidin-2-yl]methanol
[1-(4-aminophenyl)-4-hydroxymethylazetidin-2-yl]methanol
and the addition salts thereof with an acid.

6. Composition according to any one of the preceding claims, characterized in that the para-phenylenediamine derivative(s) containing an azetidinyl group, of formulae (I) and/or (II) and/or the addition salt(s) thereof with an acid, represent from 0.0005% to 12% by weight relative to the total weight of the dye composition.

7. Composition according to claim 6, characterized in that the para-phenylenediamine derivative(s) containing an azetidinyl group, of formulae (I) and/or (II) and/or the addition salt(s) thereof with an acid, represent from 0.005% to 6% by weight relative to the total weight of the dye composition.

8. Composition according to any one of the preceding claims, characterized in that it contains one or more couplers chosen from meta-phenylenediamines, meta-aminophenols, meta-diphenols and heterocyclic couplers.

9. Composition according to claim 8, characterized in that the couplers are chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxy-benzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxy-indole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and the addition salts thereof with an acid.

10. Composition according to claim 8 or 9, characterized in that the coupler(s) represent(s) from 0.0001% to 10% by weight relative to the total weight of the dye composition.

11. Composition according to claim 10, characterized in that the coupler(s) represent(s) from 0.005% to 5% by weight relative to the total weight of the dye composition.

12. Composition according to any one of the preceding claims, characterized in that it contains at least one additional oxidation base chosen from para-phenylenediamines other than compounds of formulae (I) and (II) as defined in any one of claims 1 to 4, bis(phenyl)alkylenediamines, para-aminophenols, ortho-aminophenols and heterocyclic bases, and the addition salts thereof with an acid.

13. Composition according to claim 12, characterized in that the additional oxidation base(s) represent(s) from 0.0005% to 12% by weight relative to the total weight of the dye composition.

14. Composition according to any one of the preceding claims, characterized in that the addition salts with an acid are chosen from the hydrochlorides, hydrobromides, sulphates, citrates, succinates, tartrates, lactates, phosphates and acetates.

15. Process for the oxidation dyeing of keratin fibres, characterized in that at least one dye composition as defined in any one of claims 1 to 14 is applied to the said fibres, and in that the colour is developed at acidic, neutral or alkaline pH using an oxidizing agent which is added to the dye composition just at the time of use, or which is present in an oxidizing composition which is applied simultaneously or sequentially.

16. Process according to claim 15, characterized in that the oxidizing agent present in the oxidizing composition is chosen from hydrogen peroxide, urea peroxide, alkali metal bromates, persalts, peracids and enzymes.

17. Multi-compartment device, or multi-compartment dyeing “kit”, a first compartment of which contains a dye composition as defined in any one of claims 1 to 14 and a second compartment of which contains an oxidizing composition.

18. para-Phenylenediamlne derivatives containing an azetidinyl group, of formulae (I′) and (II′) below, and the addition salts thereof with an acid: 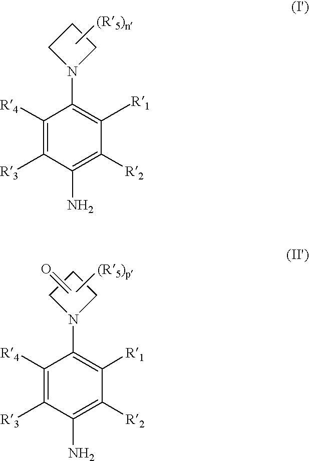
in which:
R′₁, R′₂, R′₃, R′₄ and R′₅, which may be identical or different, represent a hydrogen atom; a halogen atom; a hydroxyl radical; a C₁-₆ alkyl radical; a C₂-C₆ alkenyl radical; a C₂-C₆ alkynyl radical; a C₁-C₆ alkoxy radical; a carbamyl radical; a carboxamide radical; an N-(C₁-6) alkylcarbamyl radical; an N,N-di (C₁-C₆)-alkylcarbamyl radical; an amino radical; a (C₁-C₆)alkylamino radical; a di(C₁-₆)alkylamino radical; a (C₁-C₆)alkylcarbonyl radical; a carboxyl radical; a (C₁-C₆)alkylcarboxyl radical; a (C₁-C₆)alkylcarbonyloxy radical; a C₁-C₆ trifluoroalkyl radical; a cyano radical; a (C₁-C₆)alkylthio radical; a formyl radical; a radical CH═NHR′₆; or a 5- or 6-membered heterocycle containing from 1 to 3 heteroatoms chosen from oxygen, nitrogen and sulphur;
R′₆ represents a C₁-C₆ alkyl radical; an aromatic ring such as, for example, a phenyl ring, or a 5- or 6-membered heteroaromatic ring containing from 1 to 3 heteroatoms chosen from oxygen, nitrogen and sulphur atoms;
n′ is an integer between 1 and 4 inclusive;
p′ is an integer equal to 1 or 2;
it being understood that:
in formula (I), when n′=1 and when R′₅ represents a hydrogen atom and when one of the radicals R′₁ to R′₄ represents a substituted or unsubstituted amino radical, then at least one of the other radicals R′₁ to R′₄ is other than a hydrogen atom;
in formula (I), when n′=1, and when R′₅ represents a hydrogen atom, and when R′₂ and R′₃ simultaneously represent a hydrogen atom and when one of the radicals R′₁ or R′₄ also represents a hydrogen atom, a halogen atom, a C₁-C₆ alkyl radical, a C₁-C₆ hydroxyalkyl radical or a (C₁-6)alkoxy(C₁-C₆)alkyl radical, then the other radical R′₁ or R′₄ cannot represent a substituted or unsubstituted 5-membered heterocycle;
with the exclusion of:
4-azetidin-1-yl-3-fluorophenylamine;
3-fluoro-4-[3-(2-methoxyethoxy)azetidin-1-yl]-phenylamine;
diethyl 1-(4-aminophenyl)-2-oxoazetidine-3,3-dicarboxylate;
diethyl 1-(4-aminophenyl)-2-[1,3]dioxolan-2-yl-4-oxoazetidine-3,3-dicarboxylate;
1-(4-aminophenyl)-4-oxoazetidine-2-carboxylic acid;
methyl 1-(4-aminophenyl)-4-oxoazetidin-2-yl-methanesulphonate;
methyl 1-(4-aminophenyl)-4-oxoazetidin-2-yltoluene-4-sulphonate.

19. para-Phenylenediamine derivatives containing an azetidinyl group according to claim 18, characterized in that they are chosen from:
4-azetidin-1-ylphenylamine;
1-(4-aminophenyl)azetidine-2-carboxylic acid;
1-(4-aminophenyl)azetidine-2-carboxamide;
4-azetidin-1-yl-3-methylphenylamine;
1-(4-amino-2-methylphenyl)azetidine-2-carboxylic acid;
4-azetidin-1-yl-2-methylphenylamine;
1-(4-amino-3-methylphenyl)azetidine-2-carboxylic acid;
2-(2-amino-5-azetidin-1-ylphenyl)ethanol;
1-[4-amino-3-(2-hydroxyethyl)phenyl]azetidine-2-carboxylic acid;
2-(5-amino-2-azetidin-1-ylphenyl)ethanol;
1-[4-amino-2-(2-hydroxyethyl)phenyl]azetidine-2-carboxylic acid;
1-(5-amino-2-azetidin-1-ylphenyl)ethane-1,2-diol;
1-[4-amino-2-(1,2-dihydroxyethyl)phenyl]azetidine-2-carboxylic acid;
1-(2-amino-5-azetidin-1-ylphenyl)ethane-1,2-diol;
1- [-4-amino-3-(1,2-dihydroxyethyl)phenyl]azetidine-2-carboxylic acid;
4-azetidin-1-yl-3-dimethylaminomethylphenylamine;
1-(4-amino-2-dimethylaminomethylphenyl)azetidine-2-carboxylic acid;
4-[3-(2-methoxyethoxy)azetidin-1-yl]phenylamine;
4-[2-(2-methoxyethoxy)azetidin-1-yl]-3-methyl-phenylamine;
4-[3-(2-methoxyethoxy)azetidin-1-yl]-2-methyl-phenylamine;
1-(4-aminophenyl)azetidin-3-ol
1-(4-aminophenyl)-3-methylazetidin-3-ol
[1-(4-aminophenyl)azetidin-2-yl]methanol
[1-(4-aminophenyl)-4-hydroxymethylazetidin-2-yl]-methanol
and the addition salts thereof with an acid.

20. para-Phenylenediamine derivatives containing an azetidinyl group according to claim 18 or 19, characterized in that the addition salts with an acid are chosen from the hydrochlorides, hydrobromides, sulphates, citrates, succinates, tartrates, lactates, phosphates and acetates.

21. Derivatives according to any one of claims 18 to 20, in which n is between 1 and 3.

22. Use of the para-phenylenediamine derivatives containing an azetidinyl group, of formulae (I), (II), (I′) and (II′) as defined in any one of claims 1 to 7 and 18 and 20, as oxidation bases for the oxidation dyeing of keratin fibres.
